(19) United States
(12) Patent Application Publication
(10) Pub. No.: US 2003/0213011 A1
(43) Pub. Date: Nov. 13, 2003

(54) Insect resistant transgenic plants expressing biontin-binding protein gene

(76) Inventors: John Howard, College Station, TX (US); Michele Bailey, College Station, TX (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/275,272

(22) PCT Filed: May 17, 2001
(86) PCT No.: PCT/US01/10820

(51) Int. Cl. A01H 1/00; C12N 15/82; A01N 25/00

(52) U.S. Cl. 800/279; 424/405; 435/468

(57) ABSTRACT

Methods of controlling insect infestation of plants using a nucleotide sequence encoding biotin-binding protein to produce high levels of biotin-binding protein in the plant, i.e., a mean concentration of ≧100 ppm.

This application claims priority under 35 U.S.C. §119(e) to U.S. provisional application Serial No. 60/204,639, filed May 17, 2000.

### FIELD OF THE INVENTION

This application is directed to methods of controlling insect infestation of plants using a nucleotide sequence encoding a biotin-binding protein to produce high levels of the protein in the plant.

### BACKGROUND OF THE INVENTION

The output of world agriculture must increase if the demands of a rising population are to be met. To preserve more of what is grown, genetic engineering has been utilized to produce transgenic crops that contain proteins detrimental to critical life functions of pests and pathogens. See, e.g., Hilder and Boulter, Crop Protec. 18, 177-191 (1999). One of the first commercially successful transgenic food crops is maize, which is resistant in the field to corn borers because it contains toxins produced by the soil bacterium Bacillus thuringiensis (Bt). See, e.g., U.S. Pat. No. 5,322,687.

After harvest, the harvested plant material and commodities processed therefrom are frequently hosts to a great variety of insect and fungal pests that cause severe economic damage. Although Bt formulations have been evaluated extensively for controlling insect pests of stored grain and various dry food or feed products, no commercially-acceptable level of stored product protection against pests, especially coleopteran (beetle) pests, has been achieved. In addition, the development of insect resistance to Bt is a concern. Thus, there has been an effort to discover other insecticidal proteins.

Avidin is a glycoprotein found in avian, reptilian and amphibian egg white. The protein is composed of four identical subunits, each 128 amino acids long. The amino acid sequence has been known since 1971. See DeLange and Huang, J. Biol. Chem. 246, 698-709 (1971). The cDNA of the chicken avidin gene was documented by Gope et al., Nucleic Acid Res. 15, 3595-3606 (1987), and the genomic clone by Keinanen et al., J. Steroid Biochem. 30, 17-21 (1988). More recently, Keinanen et al., European J. Biochem. 220, 615-21 (1994), identified a family of closely related avidin genes.

Streptavidin, a protein produced by Streptomyces avidinii, was discovered in 1963 as part of an antibiotic system in culture filtrates of several species of Streptomyces. Later, Chaiet and Wolf, Arch. Biochem. Biophys. 106, 1-5 (1964), established its chemical nature and determined its amino acid composition. Streptavidin is a nearly neutral 60,000 dalton protein. It consists of 4 identical subunits each having an approximate molecular weight of 15,000 daltons. See U.S. Pat. No. 4,839,293.

Both avidin and streptavidin form a particularly strong, non-covalent bond with biotin, a vitamin of the vitamin B complex.

Nucleotide sequences encoding avidin have been introduced into plants. U.S. Pat. No. 5,962,769 is directed to the induction of male sterility in plants by the expression of avidin. The phenomenon of male sterility in avidin-producing plants was also reported in Hood et al., Molec. Breed. 3, 291-306 (1997) and U.S. Pat. No. 5,767,379.

Published PCT patent application WO 94/00992 describes the use of avidin and streptavidin as larvicides against insect pests. The application discloses applying the proteins to plants, harvested plant materials or products derived from them, or incorporating the proteins into the tissues of a susceptible plant. In the Examples, various insects or larvae were fed with an artificial diet in which avidin or streptavidin was added. The application also discloses the use of an expression cassette to introduce the genes which code for these proteins into cells of a susceptible plant species. However, only a prophetic example of such a cassette is provided, and no information whatsoever is found with regard to the concentration of expressed protein necessary to control insect infestation of the plant.

### SUMMARY OF THE INVENTION

The present inventors have surprisingly found that insect infestation of plants can be controlled by expressing a nucleotide sequence encoding a biotin-binding protein in a plant to produce mean protein concentration levels of ≧100 ppm.

Therefore, a first embodiment of the present invention is directed to a method of controlling insect infestation of a plant, by inserting into the plant's genome a nucleotide sequence encoding a biotin-binding protein, to cause expression of the sequence at a level that provides an insecticidally effective amount of the biotin-binding protein in the tissues of the plant that are normally affected by the insect, wherein the level is a mean protein concentration of ≧100 ppm.

A second embodiment of the invention is directed to a method of protecting plant tissue, harvested plant material or plant seed against infestation by a plant insect pest or larva thereof, by introducing into the plant's genome a nucleotide sequence encoding a biotin-binding protein, and expressing the sequence to produce the biotin-binding protein at a mean protein concentration that provides an insecticidally effective amount of the biotin-binding protein in plant tissue, harvested plant material or plant seed, wherein the mean protein concentration is ≧100 ppm.

A third embodiment of the invention is directed to a method of killing or slowing the growth of a plant insect pest or larva thereof, by inserting into the plant's genome a nucleotide sequence encoding a biotin-binding protein, expressing the sequence to produce the biotin-binding protein at a mean protein concentration of ≧100 ppm in plant tissue, harvested plant material or plant seed, and allowing the plant insect pest or larva thereof to consume an amount of the plant tissue, harvested plant material or plant seed sufficient to kill or slow the growth of the plant insect pest or larva thereof.

### BRIEF DESCRIPTION OF THE DRAWING

FIG. 1 shows the mortality of three species of Coleoptera (lesser grain borer, sawtoothed grain beetle and red flour beetle) as a function of avidin concentration in maize meal.

### DETAILED DESCRIPTION OF THE INVENTION

Insect infestation of plants can be controlled by expressing a nucleotide sequence encoding a biotin-binding protein in a plant to produce mean protein concentration levels of ≧100 ppm in plant tissue, harvested plant material, or plant seed. Preferably, the mean concentration levels are ≧200 ppm or ≧300 ppm, and most preferably, levels of ≧2000 ppm are achieved. A mean concentration of 100 to about 2500 ppm is suitable to control infestation by most insects, although levels exceeding 10,000 ppm can be achieved. It is useful for the plant to express the biotin-binding protein at such levels that the plant tissue, harvested plant material, or plant seed contains more biotin-binding protein than can be bound by biotin naturally occuring in the insect, preferably at least four times higher. Levels of four to eight times higher, or more, than will be bound by the biotin naturally occuring in the insect is suitable.

The phrase “biotin-binding protein” is intended to include proteins which specifically bind to biotin, such as streptavidin or avidin, derivatives or mutations of streptavidin or avidin which retain their biotin-binding ability, or combinations of these components. All forms of avidin and streptavidin derivatives and mutants are encompassed by the phrase “biotin-binding protein,” including both native and recombinant avidin and streptavidin as well as derivatized molecules, e.g., nonglycosylated avidins, N-acyl avidins and truncated streptavidins. Some of these materials are commercially available, e.g., native avidin and streptavidin, nonglycosylated avidins, N-acyl avidins and truncated streptavidin, or can be prepared by well-known methods (see Green, Methods Enzymol. 184, 51 (1990), for preparation of avidin and streptavidin; Hiller et al., Methods Enzymol. 184, 68 (1990), for preparation of non-glycosylated avidin; Bayer et al., Methods Enzymol. 184, 80 (1990) and Methods Enzymol. 184, 138 (1990), for the preparation of streptavidin and truncated streptavidin). Recombinant avidin and streptavidin can be prepared by standard recombinant DNA techniques, for example, as described by Chandra and Gray, Methods Enzymol. 184, 70 (1990), for recombinant avidin, and by Argarana et al., Nucl. Acids Res. 14, 1871 (1986), for recombinant streptavidin. See also U.S. Pat. No. 5,973,124.

The phrase “harvested plant material” is intended to include any material harvested from an agricultural or horticultural crop, including grain, fruit, leaves, fibers, or other plant parts, and products derived or obtained therefrom, such as flour, meal, or flakes derived from grain, and products in which such materials are mixed.

As used herein, the phrase “controlling insect infestation” means reducing the number of insects that cause reduced beneficial plant yield. The reduction of insect numbers can occur either through mortality, retardation of larval development (stunting), or reduced reproductive efficiency, or a combination thereof.

The plant is preferably an agricultural plant, and more preferably a cereal crop, such as maize, rye, barley, oat, millet, wheat, rice, soybean, sorghum, triticale, sunflower, alfalfa or rapeseed. However, tobacco, fiber crops such as cotton, fruit crops such as melon or tomato, and vegetable crops such as onion, pepper, cucumber, squash, carrot, crucifer (cabbage, broccoli or cauliflower), eggplant, spinach, potato or lettuce, are also included in the scope of the invention.

The insect pest to be controlled by the invention may be one which feeds upon any plant listed above, such as house fly, hide beetle, olive fruit fly, fruit fly, flour mite, European corn borer, tobacco hormworm, maize weevil, lesser grain borer, Angoumois grain moth, warehouse beetle, sawtoothed grain beetle, flat grain beetle, red flour beetle, confused flour beetle, Indianmeal moth, Mediterranean flour moth, corn rootworm, alfalfa weevil, tobacco budworm, beet armyworm, bollworm, sunflower moth, black cutworm and rice weevil.

Nucleotide sequences which code for a biotin-binding protein can be readily synthesized using automated methods, cloned, inserted into an appropriate expression cassette, and introduced directly into cells of a susceptible plant species. Accordingly, the invention includes inserting into the genome of the plant a nucleotide sequence coding for a biotin-binding protein in proper reading frame, together with transcription initiator and promoter sequences active in the plant. Transcription and translation of the sequences under control of the regulatory sequences can cause expression of the sequences at the required levels to provide an insecticidally effective amount of the protein in the tissues of the plant which are normally infected by the insect pests.

Another approach to the cloning of a biotin-binding protein nucleotide sequence employs a functional assay based on the binding of these proteins to biotin, the analysis of the isolated protein, and its “reverse engineering” to reveal the putative nucleic acid sequence. The methodology used includes identification of the gene by hybridization with probes, PCR, probe/primer/synthetic gene synthesis, sequencing, molecular cloning and other techniques which are well-known to those of ordinary skill in the art of molecular biology.

Once a biotin-binding protein nucleotide sequence has been isolated and/or engineered, it is placed into an expression vector by standard methods. The selection of an appropriate expression vector will depend upon the method of introducing the expression vector into host cells. A typical expression vector contains: prokaryotic DNA elements coding for a bacterial replication origin and an antibiotic resistance gene to provide for the growth and selection of the expression vector in the bacterial host; a cloning site for insertion of an exogenous nucleotide sequence, which in this context would code for the biotin-binding protein, eukaryotic DNA elements that control initiation of transcription of the exogenous gene, such as a promoter, and DNA elements that control the processing of transcripts, such as a transcription termination/polyadenylation sequence. The vector may also contain such sequences as necessary to allow for the eventual integration of the vector into the chromosome.

The expression vector also may contain a gene encoding a selection marker which is functionally linked to promoters that control transcription initiation. For a general description of plant expression vectors and reporter genes, see Gruber et al., “Vectors for Plant Transformation,” in METHODS IN PLANT MOLECULAR BIOLOGY AND BIOTECHNOLOGY 89-119 (CRC Press, 1993).

A promoter element employed to control expression of the biotin-binding protein, and the reporter gene, respectively, can be any plant-compatible promoter. Examples of plant gene promoters include the promoter for the small subunit of ribulose-1,5-bis-phosphate carboxylase, or promoters from the tumor inducing plasmid Agrobacterium tumefaciens, such as the nopaline synthase and octopine synthase promoters, or viral promoters such as the cauliflower mosaic virus (CaMV) 19S and 35S promoters or the figwort mosaic virus 35S promoter. See published PCT patent application WO 91/19806 for a review of illustrative plant promoters which suitably may be employed in the present invention.

The promoter that controls expression of the biotin-binding protein may be “tissue-preferred” in the sense that the expression driven by the promoter is particularly high in one tissue, while reduced expression may occur in other parts of the plant. Examples of known tissue-preferred promoters include the tuber-directed class I patatin promoter, Bevan et al., Nucleic Acids Res. 14, 4625-4638 (1986); the promoters associated with potato tuber ADPGPP genes, Muller et al., Mol. Gen. Genet. 224, 136-146 (1990); the soybean promoter of β-conglycinin, also known as the 7S protein, which drives seed-directed transcription, Bray, Planta 172, 364-370 (1987); and seed-directed promoters from the zein genes of maize endosperm, Pedersen et al., Cell 29, 1015-26 (1982).

In another embodiment, the exogenous, biotin-binding protein-encoding nucleotide sequence is under the transcriptional control of a plant ubiquitin promoter, see published European patent application 0 342 926, or the bar gene acts as the selection gene under the CaMV 35S promoter. In this construct, transcriptional activity is enhanced by a DNA fragment representing part of the CaMV 35S promoter being placed in a direct repeat tandem arrangement with the CaMV 35S promoter. See Kay et al. Science 236, 1299 (1987). The bar gene confers resistance to bialophos and to tabtoxin-β-lactam toxins. See Gordon-Kamm et al., The Plant Cell 2, 603 (1990); Uchimiya et al., Bio/Technology 11, 835 (1993), and Anzai et al., Mol. Gen. 219, 492 (1989).

In accordance with the present invention, a transgenic plant is produced that contains a DNA molecule, comprised of elements as described above, integrated into its genome so that the plant expresses a heterologous, biotin-binding protein-encoding nucleotide sequence. In order to create such a transgenic plant, the expression vectors may be introduced into protoplasts, into intact tissues, such as immature embryos and meristems, into callus cultures, or into isolated cells. Preferably, expression vectors are introduced into intact tissues. General methods of culturing plant tissues are provided, for example, by Miki et al., “Procedures for Introducing Foreign DNA into Plants,” in METHODS IN PLANT MOLECULAR BIOLOGY AND BIOTECHNOLOGY, Glick et al. (eds.), pages 67-88 (CRC Press 1993), and by Phillips et al., “Cell/Tissue Culture and In Vitro Manipulation,” in CORN AND CORN IMPROVEMENT, 3rd Edition, Sprague et al. (eds.), pages 345-387 (American Society of Agronomy 1988). The reporter gene incorporated in the DNA molecule allows for selection of transformants.

Methods for introducing expression vectors into plant tissue include the direct infection or co-cultivation of plant tissue with Agrobacterium tumefaciens, as described, for example, by Horsch et al., Science 227, 1229 (1985). Preferably, a disarmed Ti-plasmid is used as a vector for foreign DNA sequences. Transformation can be performed using procedures described, for example, in published European patent applications 0 116 718 and 0 270 822.

Other types of vectors can be used for transforming plant cells by procedures such as direct gene transfer, as described, for example, in published PCT patent application WO 85/01856 and in published European patent application 0 275 069, in vitro protoplast transformation, as described in U.S. Pat. No. 4,684,611, plant virus-mediated transformation, described in published European patent application 0 67 553 and U.S. Pat. No. 4,407,956, and liposome-mediated transformation according to U.S. Pat. No. 4,536,475, among other disclosures. Standard methods for the transformation of rice are described by Christou et al., Trends in Biotechnology 10, 239 (1992), and by Lee et al., Proc. Nat'l Acad. Sci. USA 88, 6389 (1991). Wheat can be transformed by techniques similar to those employed for transforming corn or rice. Furthermore, Casas et al., Proc. Nat'l Acad. Sci. USA 90, 11212 (1993), describe a method for transforming sorghum, while Wan et al., Plant Physiol. 104, 37 (1994), describe a method for transforming barley. Suitable methods for corn transformation are provided by Fromm et al., Bio/Technology 8, 833 (1990), and by Gordon-Kamm et al., supra.

In general, direct transfer methods are common for the transformation of a monocotyledonous plant, particularly a cereal such as rice, corn, sorghum, barley or wheat. Suitable direct transfer methods include microprojectile-mediated delivery, DNA injection, electroporation, and the like. See, e.g., Gruber et al., supra, Miki et al., supra, and Klein et al., Bio/Technology 10, 268 (1992).

The invention will be further described by the following examples.

### EXAMPLE 1

### Construction of Maize Transformed with Avidin and Preparation of Insects for Bioassays

D-Biotin and avidin were obtained from Sigma Chemical Co. (St. Louis, Mo.). Avidin was purified by Sigma from corn (catalog number A8706).

The construction of plasmids used for transformation of maize with the chicken avidin gene, transformation, tissue culture, and generation of avidin-expressing transgenic plants are described in Hood et al., supra, and U.S. Pat. No. 5,767,379. Briefly, the expression of the avidin gene was under the control of the ubiquitin promoter. The barley α-amylase signal sequence was used to secrete avidin to the endoplasmic reticulum to allow glycosylation to take place. The bar gene that codes for phosphinothricin phosphotransferase to allow resistance to the herbicide bialaphos was used as a selectable marker gene under the control of the tandem 35S promoter of cauliflower mosaic virus. The pinII terminator was used to terminate transcription. An established callus line derived from single immature embryos of the “Hi-II” corn germplasm was transformed with the avidin and bar genes by using a particle bombardment method see Tomes et al., “Direct DNA transfer into intact plant cells via microprojectile bombardment” in PLANT CELL TISSUE AND ORGAN CULTURE: FUNDAMENTAL METHODS, Gamborg and Phillips (eds.), pages 197-213 (Springer-Verlag, Berlin/Heidelberg 1995).

Transformants expressing the bar gene were selected by herbicide resistance to bialaphos. Transformants expressing the avidin gene were identified by ELISA. Rabbit antibodies to avidin were raised by purchasing purified avidin from Sigma Chemical Company (catalogue number A9275), which was sent to Bethyl Laboratories (Montgomery, Tex.) for injection into rabbits using their standard protocols. Single seeds were powdered and extracted in 10 mM phosphate buffered saline pH 7.0 containing 0.05% Tween-20. ELISAs were typical sandwich style in which the mircrotiter plates were coated with rabbit anti-avidin antibody, the avidin protein was captured overnight at 4° C., and the plate was reacted with goat anti-avidin antibody (Vector Labs, Burlingame, Calif.) followed by anti-goat alkaline phosphatase conjugate (Jackson Immunoresearch, West Grove, Pa.). The alkaline phosphatase was detected using para-nitrophenyl phosphate as the substrate and read at 405 nm on a SpectroMax plate reader (Molecular Devices, Sunnyvale, Calif.).

ELISA of extracts of individual kernels revealed that the avidin concentration was highly variable with levels ranging from 0 to approximately 2500 ppm. In one sample of 92 individual kernels, the mean concentration of avidin was 164±34 ppm. In theory, 50% of the kernels should not contain avidin because the gene was inherited from hemizygous females and not from males because nearly all of the avidin-expressing plants were male sterile.

The T₁ generation of avidin-containing seed was obtained by out-crossing the female T₀ plants with a nontransformed inbred line as the male parent. Outcrossing was performed at each subsequent generation in the field. For most of the bioassays, the T₄ generation of avidin-containing seed was used to prepare the corn meal containing 123 ppm avidin, whereas the T₅ generation was utilized for the intact kernel experiments and also for the meal that contained 265 ppm avidin.

Insects

Depending on the species, bioassays were infested with either eggs from a 24 hr period of oviposition, neonate larvae that were 0-24 hr old, or adults that were 2-4 weeks old. These were obtained from stock cultures maintained at the Grain Marketing and Production Research Center. Sitophilus zeamais (Motschulsky) (maize weevil) and Prostephanus truncatus (Horn) (large grain borer) were maintained on whole maize, whereas Rhyzopertha dominica (Fabricius) (lesser grain borer) and Sitotroga cerealella (Olivier) (Angoumois grain moth) were maintained on hard red winter wheat. Tribolium castaneum (Jacquelin du Val) (red flour beetle) and Tribolium confusum (Herbst) (confused flour beetle) were maintained on 95% wheat flour and 5% brewers yeast. Oryzaephilus surinamensis (Linnaeus) (sawtoothed grain beetle) and Cryptolestes pusillus (Schönherr) (flat grain beetle) were maintained on 90% rolled oats, 5% brewers yeast, and 5% wheat germ. Trogoderma variabile (Ballion) (warehouse beetle) was maintained on 50% rolled oats and 50powdered dog food. Plodia interpunctella (Hubner) (Indianmeal moth) and Anagasta kuehniella (Zeller) (Mediterranean flour moth) were maintained on a standard laboratory diet, see McGaughey, Science 229, 193-195 (1985).

### EXAMPLE 2

### Bioassays of Primary Pests Using Whole Kernels

Prior to bioassay, kernels were X-rayed and damaged kernels were discarded. The intact kernels were used for assays with two species of primary pests, the curculionid beetle, S. zeamais, which penetrates kernels during oviposition, and the gelechiid moth, S. cerealella, which penetrates kernels during initial larval feeding.

For S. zeamais, the kernels were equilibrated at 30° C., 12L:12D photoperiod and ˜75% RH over a saturated solution of NaCl. Three replicates of 236 g of kernels of transgenic maize (˜810 kernels) or non-transgenic maize (˜750 kernels) were infested with five female adults for 48 hr, see Throne, Environ. Entomol. 23, 1459-1471 (1994). The progeny were removed on the day that they emerged as adults. Two weeks after the last adult had emerged, the kernels were X-rayed to detect any dead larvae and pupae within the kernels. The hole that an adult had chewed during its emergence was recorded as a proof of successful infestation. It was assumed that insect damage had not significantly changed the concentration of avidin, and the concentration in each infested kernel was estimated by avidin ELISA. The number of infested kernels was one less than the total progeny (dead or alive), because two of the progeny were found in a single kernel.

After the adult females laid eggs in the kernels, individual weevil larvae developed inside many of the kernels. At the end of the bioassay, the overall mortality was significantly higher on transgenic maize than on non-transgenic maize (21±2% vs. 5±1%, respectively, P<0.01). Visual inspection and x-ray analysis identified infested and uninfested kernels. The former had either an adult emergence hole, a dead insect inside, or an oviposition (egg-laying) site. It was assumed that insect feeding did not significantly change the avidin concentration of an individual kernel and the infested kernels were subjected to avidin ELISA. Infested kernels had a mean avidin concentration of 163±27 ppm (n=3 replications, total number of kernels=149). There was approximately 7% mortality in kernels with >10 ppm avidin (78% of infested kernels), 53% mortality in kernels with 11-20 ppm (7% of infested kernels), and 96% mortality in kernels with 21-2500 ppm (15% of infested kernels). There was no mortality in kernels with low avidin (0-2 ppm) and 100% mortality in those with >300 ppm. These data indicated that overall mortality was greatly influenced by the high proportion of kernels with >10 ppm avidin. Probit analysis of the mortality for the weevil as a function of avidin concentration in individual kernels yielded LC₅₀ and LC₉₅ values of 30 and 307 ppm, respectively.

For S. cerealella, the kernels were equilibrated at 28° C., 16L:8D and ˜75% RH over a saturated solution of NaCl. Three replicates of 30 neonate larvae were placed on 20 g of transgenic maize. Emerged moths were removed daily. Two weeks after the last moth had emerged, the kernels were X-rayed to detect any dead or living larvae inside. The concentration of avidin in each infested kernel was estimated by ELISA.

Neonates bore into the kernels and typically develop inside a single kernel. Fifty-one kernels with <20 ppm avidin were found with emergence holes for adults, whereas three contained larvae that failed to pupate. Thirteen kernels with 96 to 1145 ppm avidin had larvae inside, but none of these developed into pupae. Dissection of kernels from a similar experiment revealed that approximately 30% of the infested kernels without emergence holes contained live larvae and these were probably in diapause. Probit analysis yielded EC₅₀ and EC₉₅ values of 36 and 274 ppm, respectively, for preventing the development of S. cerealella.

### EXAMPLE 3

### Bioassays of Primary Pests and Secondary Pests Using Ground Maize

Two species of bostrichid beetles, R. dominica and P. truncatus, are primary pests that can penetrate intact kernels, but they have behavioral characteristics that make them less suitable for bioassays on those kernels. The females of R. dominica lay the majority of their eggs outside the kernels, and therefore, the larval diet is often derived from more than one kernel, until each larva finally bores into a kernel to complete its development. Each female of P. truncatus bores into a kernel before laying several eggs in a side tunnel. Since it was problematic to obtain reliable information about the growth and development of these pests on kernels that contained a wide range of avidin concentrations, we utilized ground maize for bioassay of these species.

The ground maize used was from the T₄ generation, which had an avidin concentration of 123 ppm. Maize kernels were ground through a screen (20 mesh/inch) in a Wiley mill. R. dominica was bioassayed on cakes of this ground maize, because small particles of uncompacted maize were less suitable for the development of the larvae. Ground transgenic or non-transgenic maize (95) and wheat gluten (5%) were mixed with water in a 1:1.05 ratio (w/v) in a 1 oz cup (Solo Cup Co., Urbana, Ill.). A second cup was pushed down on the diet to compact it into a cake and then the cake was lyophilized. Three cakes of each type of maize were prepared. After equilibration at 28° C., 16L:8D and ˜75% RH over a saturated solution of NaCl, 50 eggs were added to each of the 2.1 g cakes. Mortalities of the immature stages were recorded from the time of infestation until all of the first generation had either died or developed into adults. For estimation of lethal concentrations of avidin, 6 control cakes were prepared using water and 3 cakes of each concentration of avidin were prepared using a solution of homogeneous avidin.

In a preliminary experiment, the mortality of P. truncatus on cakes of non-transgenic meal was excessive, since there was more than 50% mortality from the egg to the adult stage. Therefore, we used a second method to bioassay this species. The ground maize meal was compacted to a density of ˜0.7 g/ml in 0.2 ml microcentrifuge tubes. A newly hatched larva was placed in a small depression in the meal and then loosely covered with a portion of the meal. Each larva was placed on 25 mg of either transgenic meal, non-transgenic meal, or non-transgenic meal that had been slurry mixed with either water or a solution of avidin followed by lyophilization and grinding in a mortar with a pestle (2 replicates for a total of 29-30 insects).

For comparison with R. dominica, maize cakes were prepared in an identical fashion and tested with two species of secondary pests, O. surinamensis and T. castaneum, that do not attack whole kernels. The maize cakes were equilibrated at 28° C., 16L:8D, and ˜75% RH before infestation. Fifty eggs of O. surinamensis or 30 eggs of T. castaneum were placed on the cakes, and the egg-to-adult mortality was used to estimate lethal concentrations. We also estimated the lethal concentrations for isolated individuals of T castaneum. Individual eggs of T. castaneum were placed on 50 mg of maize meal in 1.5 ml microcentrifuge tubes (two replicates of ˜20 eggs). To be consistent with the analysis of the egg-to-adult mortality in a concurrent test with groups of eggs, the analysis was performed on the egg-to-adult mortality. Nearly identical estimates of lethal concentrations were obtained by analysis of larva-to-adult mortality.

Several species of secondary pests were also assayed on loose meal, because such pests normally feed on broken kernels and their larval development may be completed more quickly on loose media. Bioassays were performed with three replicates of 25 eggs of C. pusillus or 10 eggs of T. confusum or T. castaneum on 0.5 g of loose meal in 2 ml tubes. To minimize the incidence of diapause in T. variabile, which occurs more frequently when larvae are reared in isolation, two replicates of 40 eggs were placed on 8 g of meal. The pyralid moths, P. interpunctella and A. kuehniella, were assayed as isolated individuals to avoid cannibalism. Each newly hatched larva was placed on 200 mg of diet in 2.0 ml microcentrifuge tubes (2 replicates for a total of 40 larvae per treatment per species), and the lids of the tubes were pierced with a #00 insect pin to provide holes for air exchange.

When reared in groups on cakes of the maize meal, the lesser grain borer, R. dominica, sawtoothed grain beetle, O. surinamensis, and red flour beetle, T. castaneum, had mortalities of 100±0, 99±1, and 100±0%, respectively. When reared on loose meal in groups, the red flour beetle, confused flour beetle, T. confusum, and flat grain beetle, C. pusillus, had 100±0, 98±2, and 100±0% mortalities, respectively.

Control mortalities for the group bioassays were relatively high (13-31%), which was caused in part by cannibalism and the failure of some of the eggs to hatch. These variables were eliminated in the bioassays of isolated newly hatched larvae of two lepidopteran species. The mortalities of the Indianmeal moth, P. interpuntella, and Mediterranean flour moth, A. kuehniella, were 95±5 and 100±0%, respectively, on the transgenic meal, whereas the control mortalities on non-transgenic meal were 0±0 and 8±3%, respectively. With the latter species, 20±5% of the larvae grew at a normal rate on the control diet but failed to pupate after three months, indicating that they were in diapause.

With the warehouse beetle, T. variabile, we performed bioassays with ground meal from the T₄ or T₅ generation of transgenic maize containing either 123 or 265 ppm avidin. This species was assayed in groups because isolated larvae have a propensity to enter diapause. Development to the adult stage reached a maximum on meal from the non-transgenic maize by 7 weeks. On the meal from transgenic maize, none of the larvae had pupated by 16 weeks. The mortalities were 20±3, 13±5, and 65±0% at 7 weeks and increased to 23±5, 30±3, and 96±1% at 12 weeks on 0, 123, and 265 ppm avidin maize, respectively. At 16 weeks 38±8 and 100±0% mortalities occurred on 123 and 265 ppm avidin, respectively. The weight of larvae on 123 ppm avidin was 1.2±0.2 mg at 7 weeks (n=2 groups with a total of 70 larvae), 1.6±0.2 mg at 12 weeks (n=2 groups with a total of 56 larvae) and 2.5±0.1 mg at 16 weeks (n=2 groups with a total of 50 larvae). These data showed that many larvae continued to grow for several months on the 123 ppm avidin corn. Thus, the results revealed that avidin is highly toxic to the warehouse beetle at 265 ppm and that larval development is greatly suppressed at 123 ppm, perhaps due to suboptimal nutrition and diapause induction.

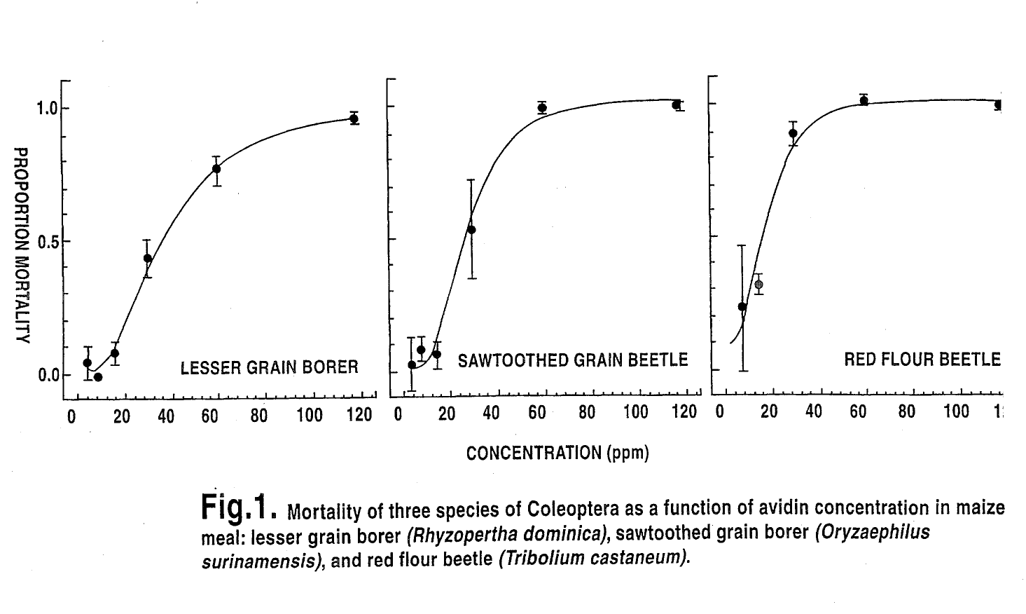

Concentration-mortality relationships were determined for the lesser grain borer, sawtoothed grain beetle, and red flour beetle using cakes of control maize meal with and without supplementation with avidin that had been purified from transgenic maize (FIG. 1). The groups that were reared on cakes with approximately 120 ppm avidin had 96-99% mortality. Probit analysis of the data provided estimates of 36 and 112 ppm for R. dominica, 29 and 63 ppm for O. surinamensis, and 19 and 47 ppm for T. castaneum for LC₅₀ and LC₉₅ values, respectively.

We performed another experiment to examine the concentration response of T. castaneum in more detail. In addition to recording the mortality of groups that were reared on cakes of meal, we recorded the mortality of isolated individuals that were reared on the loose meal and also weighed some representative individuals (n=15 to 17). The weight of the larvae fed a control diet increased from approximately 0.03 mg to 1.58±0.09 mg during 18 days following egg hatch. In contrast, a diet supplemented with 60 ppm avidin produced larvae that weighed only 0.51±0.06 mg at 18 days. The mortality of larvae fed this diet for 18 days was 13%, but this increased to 100% after 14 weeks. There was no mortality of larvae fed the control diet. The LC₅₀ was similar for individuals tested on loose meal (15 ppm with 95% confidence limits of 12-17 ppm, n=233, slope=7.73±1.70, intercept=9.13±2.16, χ²=1.9) and for groups of larvae reared on cakes of maize meal (16 ppm with 95% confidence limits of 15-18 ppm, n=570, slope=6.88±0.97, intercept=−8.36±1.27, χ²=1.7). The potency of avidin in these two concentration-response experiments was not significantly different (relative potency was 0.90 with 95% confidence limits of 0.79-1.03). These data showed that even though avidin was highly toxic to T. castaneum in a diet of maize meal, larval feeding and growth may occur for an extended period of time before death occurs.

### EXAMPLE 4

### Biotin Rescue

To determine whether exogenous biotin can prevent the detrimental effects of avidin, the vitamin was introduced as a supplement into the transgenic avidin maize meal and fed to the sawtoothed grain beetle. Ground maize was mixed with solutions of biotin in a 1:1.2 ratio (w/v). After lyophilization, the diet was ground using a mortar with a pestle. Individual eggs of O. surinamensis were placed on 20 mg of diet in 0.2 ml tubes (2 replicates for a total of 17-24 eggs that hatched), and the lids of the tubes were pierced with a pin to provide air exchange. Adult emergence was recorded daily. Individual insects were weighed to the nearest microgram using a model UMT2 Mettler balance.

Whereas the transgenic maize diet caused 100% mortality, the addition of 5 ppm biotin rescued nearly all of the insects so that the mortality was similar to that of the control group. The time from egg hatch to adult eclosion and the weight of adults for insects reared on avidin corn plus biotin were the same as those for insects fed non-transgenic corn. Thus, the toxicity of avidin maize to insects was due to biotin deprivation (avitaminosis) and supplementation of the diet with the vitamin prevented toxic and developmental effects.

Each reference cited in this application is hereby incorporated by reference in its entirety.

We claim:

1. A method of killing or slowing the growth of a plant insect pest or larva thereof, the method comprising
introducing into a plant genome a nucleotide sequence encoding a biotin-binding protein;
expressing the nucleotide sequence in the plant to produce the biotin-binding protein at a mean concentration of ≧100 ppm in plant tissue, harvested plant material or plant seed; and
allowing the plant insect pest or larva thereof to consume an amount of the plant tissue, harvested plant material or plant seed sufficient to kill or slow the growth of the plant insect pest or larva thereof.

2. The method of claim 1, wherein the nucleotide sequence encodes avidin, or a derivative or mutant thereof.

3. The method of claim 1, wherein the nucleotide sequence encodes streptavidin, or a derivative or mutant thereof.

4. The method of any one of claims 1-3, wherein the plant is maize.

5. The method of any one of claims 1-4, wherein the insect pest or larva thereof is selected from the group consisting of house fly, hide beetle, olive fruit fly, fruit fly, flour mite, European corn borer, tobacco hormworm, maize weevil, lesser grain borer, Angoumois grain moth, warehouse beetle, sawtoothed grain beetle, flat grain beetle, red flour beetle, confused flour beetle, Indianmeal moth, Mediterranean flour moth, corn rootworm, alfalfa weevil, tobacco budworm, beet armyworm, bollworm, sunflower moth, black cutworm and rice weevil.

6. The method of any one of claims 1, 4 or 5, wherein the biotin-binding protein is produced at a mean concentration of ≧100 ppm in plant tissue.

7. The method of any one of claims 1, 4 or 5, wherein the biotin-binding protein is produced at a mean concentration of ≧100 ppm in harvested plant material.

8. The method of any one of claims 1, 4 or 5, wherein the biotin-binding protein is produced at a mean concentration of ≧100 ppm in plant seed.

9. The method of any one of claims 1 or 4-8, wherein the biotin-binding protein is produced at a mean concentration of ≧200 ppm.

10. The method of any one of claims 1 or 4-8, wherein the biotin-binding protein is produced at a mean concentration of ≧300 ppm.

11. The method of any one of claims 1 or 4-8, wherein the biotin-binding protein is produced at a mean concentration of ≧2000 ppm.

12. The method of any one of claims 1 or 4-8, wherein the biotin-binding protein is produced at a mean concentration of 100 to about 2500 ppm.

13. The method of claims 1 or 5, wherein the biotin-binding protein is produced at a mean concentration of ≧100 ppm in ground maize.

14. A method of protecting plant tissue, harvested plant material or plant seed against infestation by a plant insect pest or larva thereof, the method comprising
introducing into a plant genome a nucleotide sequence encoding a biotin-binding protein; and
expressing the nucleotide sequence in the plant to produce the biotin-binding protein at a mean concentration that provides an insecticidally effective amount of the protein in plant tissue, harvested plant material or plant seed, wherein the mean concentration is ≧100 ppm.

15. The method of claim 14, wherein the nucleotide sequence encodes avidin, or a derivative or mutant thereof.

16. The method of claim 14, wherein the nucleotide sequence encodes streptavidin, or a derivative or mutant thereof.

17. The method of any one of claims 14-16, wherein the plant is maize.

18. The method of any one of claims 14-17, wherein the insect pest or larva thereof is selected from the group consisting of house fly, hide beetle, olive fruit fly, fruit fly, flour mite, European corn borer, tobacco hormworm, maize weevil, lesser grain borer, Angoumois grain moth, warehouse beetle, sawtoothed grain beetle, flat grain beetle, red flour beetle, confused flour beetle, Indianmeal moth, Mediterranean flour moth, corn rootworm, alfalfa weevil, tobacco budworm, beet armyworm, bollworm, sunflower moth, black cutworm and rice weevil.

19. The method of any one of claims 14, 17 or 18, wherein the biotin-binding protein is produced at a mean concentration of ≧100 ppm in plant tissue.

20. The method of any one of claims 14, 17 or 18, wherein the biotin-binding protein is produced at a mean concentration of ≧100 ppm in harvested plant material.

21. The method of any one of claims 14, 17 or 18, wherein the biotin-binding protein is produced at a mean concentration of ≧100 ppm in plant seed.

22. The method of any one of claims 14 or 17-21, wherein the biotin-binding protein is produced at a mean concentration of ≧200 ppm.

23. The method of any one of claims 14 or 17-21, wherein the biotin-binding protein is produced at a mean concentration of ≧300 ppm.

24. The method of any one of claims 14 or 17-21, wherein the biotin-binding protein is produced at a mean concentration of ≧2000 ppm.

25. The method of any one of claims 14 or 17-21, wherein the biotin-binding protein is produced at a mean concentration of 100 to about 2500 ppm.

26. The method of claims 14 or 18, wherein the biotin-binding protein is produced at a mean concentration of ≧100 ppm in ground maize.

27. A method of controlling insect infestation of a plant, comprising introducing into the genome of the plant a nucleotide sequence encoding a biotin-binding protein, to cause expression of the nucleotide sequence at a level that provides an insecticidally effective amount of the protein in the tissues of the plant that are normally affected by the insect, wherein the level is a mean concentration of ≧100 ppm.

28. The method of claim 27, wherein the nucleotide sequence encodes avidin, or a derivative or mutant thereof.

29. The method of claim 27, wherein the nucleotide sequence encodes streptavidin, or a derivative or mutant thereof.

30. The method of any one of claims 27-29, wherein the plant is maize.

31. The method of any one of claims 27-30, wherein the insect is selected from the group consisting of house fly, hide beetle, olive fruit fly, fruit fly, flour mite, European corn borer, tobacco hormworm, maize weevil, lesser grain borer, Angoumois grain moth, warehouse beetle, sawtoothed grain beetle, flat grain beetle, red flour beetle, confused flour beetle, Indianmeal moth, Mediterranean flour moth, corn rootworm, alfalfa weevil, tobacco budworm, beet armyworm, bollworm, sunflower moth, black cutworm and rice weevil.

32. The method of any one of claims 27-31, wherein the level is a mean concentration of ≧200 ppm.

33. The method of any one of claims 27-31, wherein the level is a mean concentration of ≧300 ppm.

34. The method of any one of claims 27-31, wherein the level is a mean concentration of ≧2000 ppm.

35. The method of any one of claims 27-31, wherein the level is a mean concentration of 100 to about 2500 ppm.
